(19) United States
(12) Patent Application Publication
(10) Pub. No.: US 2020/0207925 A1
(43) Pub. Date: Jul. 2, 2020

(54) LIGNOCELLULOSIC BIOMASS TREATMENT METHOD AND SYSTEM

(71) Applicants: The Hong Kong Polytechnic University, Kowloon (HK); Recyclegreen Project Limited, Fanling (HK)

(72) Inventors: Chengyu DONG, Kowloon (HK); Shao-Yuan LEU, Kowloon (HK); Chi Shun YEUNG, Kowloon (HK); Ho Yin TSE, Kowloon (HK); Md Khairul ISLAM, Kowloon (HK)

(73) Assignees: The Hong Kong Polytechnic University, Kowloon (HK); Recyclegreen Project Limited, Fanling (HK)

(21) Appl. No.: 16/235,851

(22) Filed: Dec. 28, 2018

(51) Int. Cl. C08H 7/00; C08H 8/00; C12P 19/02; C12P 19/14; B01D 11/02; B01D 3/00; B01D 3/14

(52) U.S. Cl. CPC C08H 6/00; C08H 8/00; C12P 19/02; B01D 3/14; B01D 11/0288; B01D 11/028; B01D 3/002; C12P 19/14

(57) ABSTRACT

There is provided a method and system of treating lignocellulose biomass for extraction of target chemical products therefrom. The method comprises pretreating the lignocellulose biomass with at least one diol in the presence of at least one acid, and optionally, water; and separating the pretreated mixture into solid and liquid products.

### FIELD

The present disclosure relates to the field of biorefinery of biomass, in particular, lignocellulosic biomass.

### BACKGROUND

Lignocellulosic biomass is a valuable resource for the production of biofuels and functional chemicals. The chemical structure of the biomass contains three main components, i.e., cellulose, hemicellulose, and lignin, of which the fractions of the three building block chemicals vary significantly among different parts and species of plants. Cellulose is a linear polymer of glucose sugar connected to each other by 1,4-glycosidic linkages and arranged in orderly fiber with crystallize and amorphous region). Hemicellulose is a branched polymer composed mainly of pentoses (such as xylose and arabinose) and hexoses (such as glucose, mannose, and galactose). Hemicelluloses act as a matrix in plant cell wall. Finally, lignin is a three dimensional aromatic biopolymer which provide physical strength to plant cell walls. Lignocellulosic structures are strong and resist physical, chemical, and biological degradations. The biomass feedstock derived from agricultural and municipal activities can be composed of different plant species (i.e., herbs, shrubs, hardwood, and softwood) and/or different parts of the plants.

Sugar platform biorefinery is a widely considered process for its high economic and environmentally friendly potential in production of biofuels and biochemicals. In this process, lignocellulosic biomass is digested into sugars with enzymes. The sugars are then transformed into desirable products via fermentation or chemical catalytic processes.

Unlike cellulose and hemicellulose, lignin is mainly composed of aromatic subunits, which are nearly non-biodegradable and hence cannot serve as a carbon source for fermentation.

Pulp/paper mills may also produce lignin, however this type of lignin is an incidental and typically underutilised by-product used only for energy recovery through direct combustion. The pulping process produces a fibrous slurry that is fed to a paper machine to produce paper. To guarantee the quality of the end products, pulping processes aim to produce strong/long cellulosic fibers, and preservation of high hemicelluloses contents in the products is preferred. The processes take place under controlled reaction conditions related to a highly specific feedstock selected so as to maximise the cellulose yield for papermaking.

However, with more attention to “green”, “environmentally-friendly” chemistry, lignin has been reconsidered as a valuable chemical if it can be properly harvested or processed. As the only renewable aromatic compounds naturally occurring, lignin has superior functionality in UV absorbance, anti-oxidation, amphiphilicity, low cytotoxicity (Ten, E., et al., Lignin Nanotubes As Vehicles for Gene Delivery into Human Cells. Biomacromolecules, 2014. 15(1): p. 327-338) and can be controlled by pH/solvent modifications (Tian, D., et al., Lignin valorization: lignin nanoparticles as high-value bio-additive for multifunctional nanocomposites. Biotechnology for Biofuels, 2017. 10).

Lignin and the related derivatives have been applied in adhesives, green composites, drugs delivery and bioimaging (Chen, N. S., L. A. Dempere, and Z. H. Tong, Synthesis of pH-Responsive Lignin-Based Nanocapsules for Controlled Release of Hydrophobic Molecules. Acs Sustainable Chemistry & Engineering, 2016. 4(10): p. 5204-5211; Dai, L., et al., Lignin Nanoparticle as a Novel Green Carrier for the Efficient Delivery of Resveratrol. Acs Sustainable Chemistry & Engineering, 2017. 5(9): p. 8241-8249; Dongre, P., et al., Lignin-Furfural Based Adhesives. Energies, 2015. 8(8): p. 7897-7914; Faris, A. H., et al., Combination of lignin polyol-tannin adhesives and polyethylenimine for the preparation of green water-resistant adhesives. Journal of Applied Polymer Science, 2016. 133(20); Thakur, V. K., et al., Progress in Green Polymer Composites from Lignin for Multifunctional Applications: A Review. Acs Sustainable Chemistry & Engineering, 2014. 2(5): p. 1072-1092).

With its biocompatible and environmentally-friendly nature, lignin-based nanoparticles have showed potential as functional group carriers (Yiamsawas, D., et al., Biodegradable lignin nanocontainers. Rsc Advances, 2014. 4(23): p. 11661-11663; Qian, Y., et al., CO₂-responsive diethylaminoethyl-modified lignin nanoparticles and their application as surfactants for CO₂/N-2-switchable Pickering emulsions. Green Chemistry, 2014. 16(12): p. 4963-4968; Zhao, W. W., et al., From lignin association to nano-/micro-particle preparation: extracting higher value of lignin. Green Chemistry, 2016. 18(21): p. 5693-5700). Lignin monomers are increasingly recognized as an essential precursor for producing renewable aromatic chemicals.

Pretreatment is an important step to fractionate cellulose, hemicellulose, lignin, and extractives from the plant cells of biomass processed in a biorefinery. Pretreatment direct impacts the structural and chemical compositions of lignocellulosic biomass, and increases the accessibility of cellulose (Alvira, P, et al., Pretreatment technologies for an efficient bioethanol production process based on enzymatic hydrolysis: a review. Bioresource technology 101, no. 13 (2010): 4851-4861), hemicellulose, and lignin. The key steps of pretreatment include hydrolysis of hemicellulose, dissociation and extraction of lignin, reduction of polymer sizes of the substrate, and reducing crystallization of cellulose. These can enhance enzymatic saccharification of lignocellulosic biomass and fractionate lignin for lignin to aromatic monomer conversion. Pretreatment of lignocellulosic biomass can be accomplished by heating, steam explosion, acid, base or organosolv processes.

Steam explosion pretreatment has been successfully applied on a wide range of lignocellulosic biomasses with or without chemical addition; but this process needs to be carried out at high temperature (i.e., approximately 220° C.). The structure of lignocellulosic biomass can be loosened due to significant shearing forces and heat, resulting in physical separation of fibers, hemicellulose removal, melting and partial depolymerisation of lignin. However, the pretreatment is not effective in dissolving lignin and hence the majority of the biomass lignin fraction will also be separated together with other insolubles. The efficiency of saccharification is affected due to non-productive adsorption of the enzyme (cellulase) on the lignin.

To reduce the operation temperature, acids (i.e., acetic acid, formic acid, sulfuric acid, hydrogen chloride, or sulfur dioxide) and/or bases (i.e., sodium hydroxide) have been used in pretreatment processes to catalyze the dissociation of plant cell walls. Based on the physiochemical characteristics of the biomass the operation temperature of the pretreatment processes can varied between 120 and 180° C.

Liquid ammonia, instead of dilute acid, has been applied to effectively reduce the lignin fraction of the lignocellulosic materials. However, during ammonia fiber explosion pretreatment (AFEX) a part of the phenolic fragments of lignin and other cell wall extractives can still remain on the cellulosic surface. AFEX pretreatment does not significantly solubilize hemicellulose if compared with dilute acid pretreatment. Furthermore, ammonia must be recycled after the pretreatment in order to reduce cost and protect the environment.

Additionally, the harsh reaction environment of these conventional pretreatment processes (e.g., steam explosion and dilute acid/alkaline processes) can lead into undesired irreversible lignin degradation and condensation, especially when the biomass feedstock is formed by a mixture of different plant species. Difficulties arise from monitoring, adjusting and optimizing the pretreatment conditions for different plant species. In organosolv processes, lignocellulose is mixed with an organic solvents/water solution and heated to dissolve the lignin and part of the hemicellulose, leaving reactive cellulose in the solid phase. A variety of organic solvents such as alcohols, esters, ketones, glycols, organic acids, phenols, and ethers have been considered in this process. For economic reasons, low-molecular-weight alcohols such as ethanol and methanol have been in favor in organosolv pretreatments. In organosolv pretreatment processes lignocellulosic biomass is fractionated into a cellulose-enriched solid product stream (pulp) and a liquid product stream (liquor) comprising dissolved lignin and hemicellulose derivatives. Pretreatments using organic solvents have high selectivity which would not destroy cellulose while preserving high purity lignin for further utilization. However, the extent of delignification in organosolv pretreatment relies on the effective cleavage of ether linkages, which is detrimental to lignin-to-aromatic conversion processes. Due to concerns potentially related to feedstock logistic and storage, infrastructure and manufacture cost, particularly, the price of ethanol, ethanol pretreatment has not yet been applied commercially. Furthermore, known ethanol pretreatment methods require an operating temperature of 155 to 198° C. (Pan, X. et al., Biorefining of softwoods using ethanol organosolv pulping: Preliminary evaluation of process streams for manufacture of fuel-grade ethanol and co-products. Biotechnol. Bioeng., 2005. 90: 473-481; Pan, X. et al., Bioconversion of hybrid poplar to ethanol and co-products using an organosolv fractionation process: Optimization of process yields. Biotechnol. Bioeng., 2006. 94: 851-861).

High-boiling solvent (HBS) pulping is a organosolv process for paper manufacture using aqueous high boiling point diols as an organic solvent for pretreatment, such as 1,3-butanediol (i.e., 1,3-BDO with a boiling point, b.p. of 208° C.) and 1,4-butanediol (1,4-BDO, b.p.=232° C.). In the pulping process the feed stock and reaction conditions are carefully controlled for removal of lignin to obtain the maximum cellulose yield in the pulp (fibrous slurry) used for papermaking. Kishimoto et al. reported that both hardwoods and softwoods can be delignified extensively at 200° C. and 220° C., respectively (Kishimoto et al., Delignification mechanism during high-boiling solvent pulping part 1. reaction of guaiacylglycerol-β-guaiacyl ether, Holzforchung, 2001, 55, p 611-616). Ether linkages of lignin are cleaved homolytically via quinone methide intermediates under HBS pulping conditions, which eliminate the formation of Hibbert's ketones.

However, the HBS process for paper production takes place at 200° C. or above and requires the use of reactors that can withstand high pressure. In particular, the reactor used in the HBS process for pulping is typically made of thick, high quality stainless steel, e.g., 316SS. The use of catalyst (e.g. acid) at such high temperature would break cellulose into smaller pieces, affecting the quality of the paper, the highly efficient production of which is the key outcome of the pulping process.

Despite the increasing environmental pressure favouring switching from petrochemical refinery production to biorefinery production, there are a number of challenges involved with the generation of biofuels and useful products from biomass in an economic and sustainable manner. Therefore, biorefinery processes remain largely conceptual and real life commercial exploitation is limited.

Typically, the known processes require optimization of pretreatment conditions for different biomass species, which means that a single processing system cannot effectively perform the biorefinery process for a range of biomass materials (e.g. hardwood, softwood, rice straw, wheat straw, bagasse) or mixed biomass materials (e.g. mixtures of different wood species, mixtures of softwood and hardwood).

The use of environmentally unfriendly technologies, toxic solvents, high energy (e.g. use of high temperature), and high costs (and high profile reactor) in the biorefinery technologies make it difficult to produce biofuels in a manner that is commercially viable and profitable. Therefore, the replacement oil refinery products with biofuels remains to be hindered by these challenges.

It is an object of the present disclosure to provide an improved biorefinery process that ameliorates or addresses at least some of the above deficiencies.

### SUMMARY

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims.

In accordance with a first aspect of the present disclosure, there is provided a method of treating lignocellulose biomass for extraction of target chemical products therefrom comprising: pretreating the lignocellulose biomass with at least one diol in the presence of at least one acid, and optionally, water; and separating the pretreated mixture into solid and liquid products.

In an embodiment, the pretreatment step is performed at or below about 170° C. Preferably, the pretreatment step is performed at a temperature range of between about 100° C. to about 170° C., preferably slightly above (i.e., 200 kPa) or under atmospheric pressure.

In an embodiment, the lignocellulosic biomass is selected from the group comprising grasses, straws, husk, bagasse, wheat, hardwood, softwood, and combinations thereof. In an exemplary embodiment, the lignocellulosic biomass is comprised of a plurality of plant species.

Advantageously, the at least one diol used in the disclosed method is selected from the group consisting of ethylene glycol, 1,2-propanediol, 1,3-propanediol, 1,3-butanediol, and 1,4-butanediol. In a preferred embodiment, the at least one diol is 1,4-butanediol.

Advantageously, the at least one acid used in the disclosed method is selected from the group consisting hydrochloric acid, formic acid, acetic acid and phosphoric acid and sulphuric acid. In a preferred embodiment, the at least one acid is sulfuric acid, and the concentration of acid may be in the range of 1 mM-200 mM. In an exemplary embodiment, the ratio of diol to biomass is 1:7 or lower, and the ratio of water to diol is 1:100 or lower.

The presently disclosed method may further comprises the steps of extracting lignin from the liquid product, enzymatic hydrolysis of the solid product, and optionally fermentation of the sugars obtained from the hydrolysis. For example, enzymatic hydrolysis may be carried out using one or more enzymes selected from the group consisting of cellulose, endo-cellulase, exo-cellulase, beta-glucosidase, cellobiase, oxidative cellulase, cellulose phosphorylases, hemicellulases, xylanases, arabinases, mannanases. Optionally, the presently disclosed method may further comprises the step of fractional distillation of the fermentation product to recover target products.

Advantageously, the pretreatment step, the enzymatic hydrolysis step and the fermentation step of the presently disclosed method can take place in the same reactor. In accordance with a second aspect, there is provided a system for treating one or more lignocellulose biomass. The system comprises a reactor for carrying out a pretreatment step involving contacting lignocellulose biomass with at least one diol in the presence of at least one acid. In a preferred embodiment, the reactor is made of a material selected from plastic, glass and metal.

In an embodiment, the presently disclosed system further comprises one or more of the following: means for separating the pretreated mixture to produce a cellulose-enriched product stream and lignin-enriched product stream; means for size reduction; means for separating purified lignin; a saccharification and fermentation unit; and a fractional distillation unit.

Advantageously, the system presently disclosed comprises a single unit for pretreatment, enzymatic hydrolysis and fermentation.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings.

Preferred embodiments of the present disclosure will be explained in further detail below by way of examples and with reference to the accompanying drawings, in which:

FIG. 1 illustrates an exemplary possible system for performing a diol based biorefinery process according to embodiments of the present technology. As depicted, the system for treating biomass (9) comprises one or more of the following units: (a) an first operation unit (e.g., hammer mill) (10) for size reduction of the biomass mater; (b) a pretreatment reactor (12); (c) separation unit (14) for solid/liquid separation for producing a cellulose-enriched product stream and lignin-enriched product stream (liquor); (d) second operation unit for size reduction (e.g. disc milling or grinding) (16); (e) separation unit (18) for separating purified lignin (17), e.g. by anti-solvent treatment; (f) saccharification and fermentation unit (20); and (d) fractional distillation unit (22) for producing fermentation products (19). In an exemplary embodiment, units 12 and 20 can be replaced by a one single reactor operating under a sequencing batch mode.

FIG. 2 showed the vapor pressure in various temperature for conventional pretreatment solvent.

FIG. 3 shows the chemical compositional analysis of organosolv pretreated substrate. Boxed groups show consistently low lignin contents of the pretreated biomass after diol pretreatment with different severities. Lignin condensation occurs in other organosolv in FIG. 3 (b).

FIG. 4 illustrates 2D HSQC NMR spectra of EtOH (left) and 1,4-BDO (right) residual lignin.

FIG. 5 illustrates 2D HSQC NMR spectra of EtOH and 1,4-BDO dissolved lignin.

FIG. 6 illustrates aliphatic OH and phenols contents of organosolv soluble lignin.

FIG. 7 illustrates enzymatic digestibilities of organosolv pretreated Eucalyptus.

### DETAILED DESCRIPTION OF THE PREFERRED EMBODIMENTS

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

The disclosed technology addresses the need in the art for a biorefinery process can be used in a commercial scale in an economical manner. A novel approach is described herein to provide a method for the treatment of lignocellulose-containing biomass to produce organic products and energy. The disclosed method is based on modified organosolv process, including a pretreatment step involving the use in combination of at least one diol and at least one acid catalyst. Unexpectedly the presently disclosed diol/acid pretreatment process is able to dissolve a significantly large amount of lignin from diverse lignocellulose-containing biomass at a moderate temperature (under 170° C. or even lower) in an efficient manner. As shown by the examples (Example 3), the presently disclosed process is biomass non-specific, meaning that it can be used for the production of useful organic products from a diverse source of lignocellulose-containing biomass, e.g. a mixture of waste wood. This significantly increases the number of applications, making this process especially suitable for use in waste management (where the source of biomass typically comprises biomass wastes from many different plant species mixed together). Using this process means the same industrial plant can be used for the treatment of various biomass substrates.

Additionally, the presently disclosed method and system is demonstrated to have an increased yield and efficiency over existing processes. The process of the present disclosure does not create stringent pretreatment conditions (less lignin degradation and condensation), and furthermore is able to be applied to a diverse range of biomass, making it a highly desirable biomass refinery process.

It is envisaged that the presently disclosed biorefinery system allows various steps of biomass treatment (pretreatment, hydrolysis, and fermentation) to be carried out in a single reactor (as illustrated by the use of a glass bottle), thereby reducing the number of operation units, or even eliminating the need for separate units.

Referring to the drawings, there is shown an exemplary system for performing a diol based biorefinery process of the present technology. In an embodiment, the selected lignocellulose-containing biomass is reduced in particle size at an operation unit (10), e.g. by a hammer mill to obtain biomass material of a suitable size. For example, the untreated biomass (9) may be chopped or milled to pieces for ease of handling (e.g., pieces of 9 to 50 mm, in particular 5-8 mm in thickness, depending on the type of biomass).

### Pretreatment Step

The present biomass processing method and system may be understood by making reference to the drawing in FIG. 1, which shows one embodiment of the system. In reference to FIG. 1, in an exemplary embodiment, the lignocellulose biomass is treated with at least one diol in the presence of at least one acid at a pretreatment reactor (12) (e.g. a vessel made of steel, glass or plastic) in order to separate the biomass into a cellulose-enriched product stream and a lignin-enriched product stream (liquor). Advantageously, the diol pretreatment is performed at a temperature below 170° C., preferably between 100° C. and 170° C., more preferably between 120° C. and 170° C. and even more preferably between 120° C. and 150° C. In an embodiment, the diol pretreatment is performed at a temperature between 100° C. and 120° C. The diol pretreatment step may be performed under atmospheric pressure, where the pressure inside the reactor is not higher than the pressure of the outer environment. The duration of the diol pretreatment may be any time suitable to effectuate the fractionation, as will be appreciated by the skilled person. Typically, the diol pretreatment step is performed for 30 to 250 minutes, preferably 30 to 150 minutes, more preferably 30-120 minutes, and most preferably 30-60 minutes.

The pretreatment solution comprises at least one diol solvent. Diols are compounds comprising two hydroxyl groups (OH group), and preferred examples of diol are ethylene glycol, 1,2-propanediol, 1,3-propanediol, 1,3-butanediol, 1,4-butanediol. In a preferred embodiment, a catalytic amount of acid is included in the pre-treatment solution. Acid is present to lower the pH. Typically, the pH during diol pretreatment is between 0.5 and 7.0, preferably between 1.5 and 3.0. The concentration of the acid in the pretreatment solution is between 1 mM and 200 mM, more preferably between 15 mM and 100 mM. Amounts of acid in the pretreatment solution above the upper limits may result in more side-reactions, and thus increase the formation of impurities. Lower amounts of acid, on the other hand, reduce the extent of fractionation and delignification realized by the present process. Suitable acids that may be used in the pretreatment steps include hydrochloric acid, formic acid, acetic acid and phosphoric acid and sulphuric acid. Preferably, the acid catalyst is sulphuric acid.

In an embodiment of the present technology, the suspension of biomass and pretreatment solution containing an acid catalyst is obtained by mixing at most 50 L and at least 0.1 L of pretreatment solution per kg dry weight of the biomass, preferably between 1.0 L and 10 L.

The optimum ratio of treatment liquid to biomass depends on the type of biomass and can be readily adjusted by those skilled in the art. For economic reasons, the liquid to solid weight ratio (L/S) of the diol pretreatment is preferably as low as possible, preferably at or lower than 7/1.

Advantageously, the pretreatment solution is further mixed with water in the pretreatment vessel (12). The presence of water in the pretreatment solution allows hydrolysis reactions to take place when the network of structural components is being broken up by diols. It is understood that hydrolysis takes place at the covalent bonds between e.g. lignin and hemicellulose, and not necessarily between the glucose monomers within a polysaccharide chain such as cellulose. Preferably, the pretreatment solution comprises at least 1 wt % water, more preferably at least 10 wt %. In other words, the water/diol ratio is 1/100 or less.

The pretreated lignocellulosic biomass is separated into a cellulose-enriched product stream and a lignin-enriched product stream (liquor) by any separation method known to the person skilled in the art, for example but not limited to: pressing, filtration, sedimentation or centrifugation at the separation unit (14). The diol/acid pretreatment step of lignocellulosic biomass results in a cellulose-enriched extract containing less impurities and a lignin-enriched liquor containing a higher content of lignin.

### Enzymatic Hydrolysis

Enzymatic hydrolysis of cellulose to glucose is accomplished by an enzyme or combination of enzymes capable of hydrolyzing cellulose, and these enzymes are referred to as hydrolytic enzymes, preferably cellulases. Hydrolysis of cellulose is also known as cellulolysis. The process according to the present technology may be performed using any cellulase enzyme.

Suitable cellulase enzymes are endo-cellulases (cleaving cellulose at inner positions), exo-cellulases (cleaving cellulose at more external positions to produce cellobiose or cellutetraose), beta-glucosidases (cellobiases, cleaving the exocellulase products intoglucose units). Other cellulase enzymes, such as oxidative cellulases and cellulose phosphorylases, are less preferred. Preferably a combination of cellulase enzymes is used, in particular a combination of endo-cellulase, exo-cellulase and β-glucosidase. Also, hemicellulases (e.g. xylanases, arabinases, mannanases, etc.) may be present to decompose any residual hemicellulose remaining after the diol pretreatment although hemicelluloses have been completely dissolved in the pretreatment liquor in the given examples.

In an exemplary embodiment of the present technology, the cellulose-enriched product stream obtained as a product from the diol pretreatment step, is subjected to enzymatic hydrolysis at the saccharification and fermentation unit (16) at FIG. 1. Optionally, the cellulose-enriched product stream is subjected to disc milling or grinding (with additional water and pH modification) at a second operation unit (16) (d) prior to enzymatic hydrolysis. Disc milling or grinding are commonly employed defiber processes to produce shorter cellulose fibers, which are advantageous for the downstream saccharification and fermentation processes. Optionally, water is added to modify the pH (e.g. to a pH range of 2.0-7.0, preferably 4.0-6.0, more preferably pH 4.8) before hydrolysis and fermentation of sugars.

### Further Process Steps

In an exemplary embodiment of the present technology, the liquor obtained from separating off the cellulose-enriched stream, may be further treated or separated for the purpose of isolating other valuable products.

In an embodiment, lignin from the lignin-enriched product stream (liquor) is subjected to further solid/liquid separation. Specifically, the liquor obtained at the separation unit (14) contains appreciable levels of lignin, hemicellulose derivatives, e.g. xylose and its oligomers and polymers, organic acids, salts and other compounds.

Lignin (17) can be obtained at solid/liquid separation unit (18) by precipitation by decreasing the organic solvent content in the liquor by the addition of anti-solvent, e.g. water to precipitate out the water insoluble polymer, and/or by evaporation of (non-hydroxylic and/or other) organic solvent, followed by centrifugation.

The aqueous filtrate, which is depleted in lignin and contains appreciable levels of hemicellulose derivatives, e.g. xylose and its oligomers and polymers, may be subjected to further process steps for recovering or valorizing these carbohydrates with commercially available processes, e.g. by anaerobic treatment using an anaerobic culture from commercial anaerobic digestions or fermentations as a starting sludge. One or more downstream processing steps are optional and not shown in FIG. 1.

In an embodiment, the system further comprises a fractional distillation (22) for producing purified final organic products from the fermentation products (19), e.g. ethanol, butanol, lactic acid. Optionally, the treatment process further comprises of recovering and recycling solvents through evaporation and condensation.

### Biomass

Conventional biomass treatment methods typically are biomass-specific, therefore complex optimization processes are required to treat different biomass types, such as wood from different tree species. However, as demonstrated herein, diol solvent in combination of acid is able to effectively dissolve lignin from various biomass species, allowing complex feedstocks to be effectively treated. Therefore, it is envisaged that any suitable type of biomass can be used in the present technology.

Suitable lignocellulosic biomass that can be processed utilizing the disclosed method include forestry residues, agricultural residues, yard waste, animal and human waste (e.g., biodegradable municipal waste). For example, the biomass can be herbaceous biomass, including grasses, straws (e.g. rice straw, wheat straw), husk (rice husk, wheat husk), bagasse, hardwood, softwood, and/or combination of these feedstocks. Optionally, prior to the pretreatment step, the untreated biomass can be washed and/or reduced in size, e.g. by chopping, chipping, debarking, milling, or grinding.

The ability of the diol/acid pretreatment method is particularly useful for treating woody biomass waste, which is typically composed of mixed plant species, with varying amounts and types of lignin. With this method, yard and woody waste can be converted to liquid fuels in a cost effective manner.

### Pretreatment Conditions

Due to the harsh biomass treatment conditions, conventional biomass treatment methods often require the use of a pressure vessel made of materials that withstand high temperatures and pressures, such as carbon steel or stainless steel or similar alloy.

For example, due to high vapor pressure of ethanol at desired temperature (i.e., 120 to 170° C.), a high-profile pressure vessel (e.g. typically made of stainless steel with thickened walls) is required for to perform ethanol pretreatment and the pulping process of Kishimoto. Another example is acid/base treatment of biomass which is performed at 170° C. under high pressure of >600 kPa.

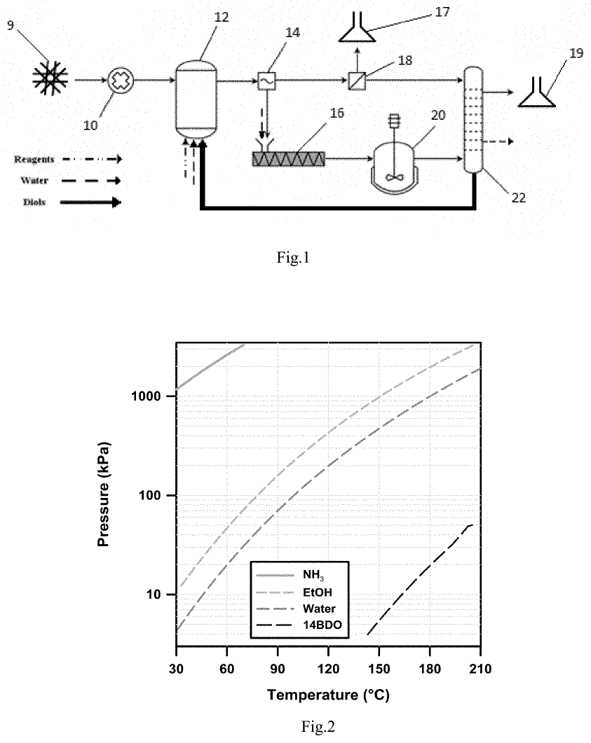

On the other hand, as compared with water, and ethanol that are used in conventional pretreatment methods, diols have higher boiling points (the boiling point of 1,4-BDO b.p. 230° C.) and lower vapor pressure in general. FIG. 2 showed the vapor pressure in various temperature for conventional pretreatment solvent. The vapor pressure of 1,4-BDO at 121° C. is approximately 8 kPa (in comparison with approximately 200 kPa for water and 300 kPa for ethanol), therefore, allowing diol/acid pretreatment to be carried out using a low-profile reactor, as confirmed using a serum bottle at simple laboratory settings. The presently disclosed pretreatment step can be carried out in a reactor made of plastic, glass, or stainless steel, e.g. with only 1-5 mm thickness, preferably 3-5 mm thickness, and more preferably, 3-4 mm thickness. Advantageously and for example, the pretreatment step can be performed in commercial fermenter tanks (e.g. with 5 mm 316SS) without modification.

This special feature allow sequencing batch reactor mode to be feasible in a biorefinery, allowing batch processing of biomass. With the presently disclosed method, a first generation biorefinery (using food or starchy feedstock for biofuel production) can be easily upgraded to a second generation biorefinery (using agricultural and forestry residues as feedstock) without converting the cooking unit to high pressure vessel (to hold >300 kPa). Therefore, the use of diol/acid pretreatment and reduce the number of operation parts in a biomass treatment system, reducing overall costs.

### Downstream Products

As further discussed in the example section herein, the presently disclosed treatment process enhances the overall lignin extraction yield from the lignocellulose biomass. Whilst the typical extraction yield with conventional processes is about 80%, diol pretreatment can dissolve lignin effectively and fractionates 90% of reactive lignin. Therefore, a 90% extraction yield of lignin with higher quality is made possible using the diol/acid pretreatment, at a moderate temperature. Moreover, the obtained reactive lignin has more β-O-4 linkage and less condensed phenolic groups which increases lignin-to-monomer yield. Furthermore, since the extracted lignin has a new hydroxyl group, the solubility and reactivity of dissolved lignin is increased, making it exceptionally useful in the preparation of valuable products in bio-medical and chemical fields.

A problem with the conventional pretreatment method is that significant lignin precipitation is observed when biomass is treated at severe conditions (e.g. under highly acidic pH range or at high temperatures). Any cellulose released from the biomass would be trapped by the lignin precipitates, making it unavailable for enzymatic hydrolysis and reducing the enzymatic glucose conversion yield. This problem is overcome by the presently disclosed diol/acid pretreatment, due to the effective dissolution of lignin by the pretreatment solvent. In particular, the pretreatment solution is able to maintain the dissolved lignin in the liquor.

### EXAMPLES

Aspects of the present disclosure demonstrated by the following examples that are demonstrative only and not intended to be limiting.

### Example 1—Pretreatment of Eucalyptus Chips

Materials Eucalyptus chips was provided by Leizhou Forestry Bureau (Guangdong, China).

Pretreatment solvents and other chemicals were purchased from J&K Acros Organics (Beijing, China). Commercial cellulase Cellic Ctec2 was generously provided by Novozymes Investment Co. Ltd. (Beijing, China) and Pronase was obtained from Sigma Chemical Company (USA).

### Pretreatment

Eucalyptus chips were pretreated in aqueous solvents monohydric alcohols, and diols previously pretreated with sulfuric acid as a catalyst using a custom-built, eight-vessel (500 ml each) rotating digester made by Xian Yang Tong Da Light Industry Equipment Co. Ltd. (Shanxi, China).

The tested solvents can be classified into three types, these being: (i) monohydric alcohols (methanol (MeOH), ethanol (EtOH), 2-Propanol (2-ProOH), 2-Butanol (2-BuOH); (ii) diols (ethylene glycol (EG), 1,2-propanediol (1,2-PG), 1,3-propanediol (1,3-PG), 1,3-butanediol (1,3-BDO), 1,4-butanediol (1,4-BDO)). Monohydric alcohols are known organosolv solvents for pretreatment lignocellulosic biomass, and these reagents are being tested for comparison.

A 50.00 g (oven-dried weight) batch of wood powder was mixed with 15 mM to 25 mM sulfuric acid and 65%-85% (v/v) organosolv reagents at a liquid-to-solid ratio of 7:1. The digester was heated to 170±3° C. at a rate of 3° C. per minute and maintained at the temperature for 60 min.

TABLE 1
Pretreatment parameters use in the pretreatment step

| Experiment | Pretreatment | Acid | Solvent |
|---|---|---|---|
| Condition | solvents | charge | percentage |
| 1 | MeOH | 20 mM | 65% |
| 2 | EtOH | 20 mM | 65% |
| 3 | 2-ProOH | 20 mM | 65% |
| 4 | 2-BuoH | 20 mM | 65% |
| 5 | EG | 20 mM | 65% |
| 6 | 1,2-PG | 20 mM | 65% |
| 7 | 1,3-PG | 20 mM | 65% |
| 8 | 1,3-BDO | 20 mM | 65% |
| 9 | 1,4-BDO | 20 mM | 65% |
| 10 | MeOH | 25 mM | 85% |
| 11 | EtOH | 25 mM | 85% |
| 12 | 2-ProOH | 25 mM | 85% |
| 13 | 2-BuoH | 25 mM | 85% |
| 14 | EG | 25 mM | 85% |
| 15 | 1,2-PG | 25 mM | 85% |
| 16 | 1,3-PG | 25 mM | 85% |
| 17 | 1,3-BDO | 25 mM | 85% |
| 18 | 1,4-BDO | 25 mM | 85% |

After pretreatment, the substrate and spent liquors were then separated using a nylon cloth. The substrate was washed three times (75 mL each) with warm (60° C.) aqueous organosolv solution with the same concentration as the pretreatment liquor. The spent liquor were then combined and mixed with three volumes of water (1:3 spent liquor/water ratio) to precipitate the dissolved lignin. The substrate was then washed three times with water at 60° C., and the washes were discarded.

### Example 2—Chemical Compositional Analysis of Eucalyptus

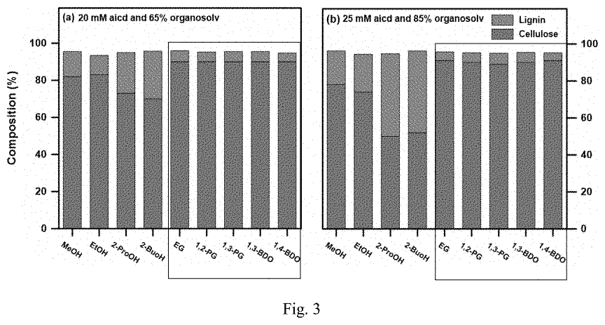

The composition of Eucalyptus after pretreatment were determined according to the procedures established by the National Renewable Energy Laboratory (NERL). The hydrolyzed monomeric sugar units were quantified via high-performance liquid chromatography (HPLC, Shimadzu) equipped with CHO-782 Transgenomic column and acid insoluble lignin content was calculated gravimetrically. The chemical compositions of organosolv pretreated substrates were presented in FIG. 3. FIG. 3 demonstrates the levels of lignin dissolution in organosolv pretreatment at two pretreatment conditions, i.e., 20 mM acid and 65% organosolv and 25 mM acid and 85% organosolv, respectively. Original Eucalyptus prior to pretreatment comprises 42% cellulose, 21% hemicellulose and 25% lignin.

As shown in FIG. 3, in the Eucalyptus treated with the comparative examples EtOH, 2-ProOH, 2-2-BuOH (i.e., experiment conditions 1-4 and 10-14), a larger amount of lignin residues are presented in the pretreated substrate due to lignin condensation (as hemicelluloses have been completely removed in all the tested processes). The lignin residues in the substrate are undesirable as bioethanol productivities in the downstream processes would be affected. On the other hand, in the Eucalyptus treated with the diols (i.e., boxed, experiment conditions 5-9 and 15-19), dissolution of lignin is considerably more effective, resulting in a significantly smaller amount of residual lignin in the substrate. In particular, the lignin contents were reduced from 25% to 4.3% to 6.0% as shown in Table 2.

The results show that, in the presence of both 20 mM acid and 25 mM acid, the tested diols isolated/dissolved more lignin as compared to monohydric alcohols (in order, methanol, ethanol, 2-ProOH, and 2-BuOH) and this difference was more significant with the increase of pretreatment severity (i.e. when harsher pretreatment condition is used). Residual lignin contents of 2-ProOH and 2-BuOH pretreatment were found to be more than 20% at elevated pretreatment severity (25 mM acid, 85% organosolv). On the other hand, residual lignin contents of diols (i.e. EG, 1,2-PG, 1,3-PG, 1,3-BDO, 1,4-BDO) pretreatment were found to be 6.0% or below, indicating that the extraction yield of lignin is over 90%. These results suggested that the lignin with changed chemical properties after pretreatment could still be dissolved in diols.

TABLE 2
Lignin content of eucalyptus different pretreatment processes

| Experiment | Lignin content | Lignin removal |
|---|---|---|
| Condition | (%) | (%) |
| 1 | 13.5% | 81.5% |
| 2 | 10.5% | 82.4% |
| 3 | 22.0% | 65.4% |
| 4 | 25.8% | 61.4% |
| 5 | 6.0% | 90.4% |
| 6 | 5.3% | 93.2% |
| 7 | 5.5% | 94.0% |
| 8 | 5.5% | 93.7% |
| 9 | 4.8% | 93.6% |
| 10 | 18.3% | 72.1% |
| 11 | 20.5% | 70.0% |
| 12 | 44.8% | 34.0% |
| 13 | 44.3% | 31.7% |
| 14 | 4.8% | 94.2% |
| 15 | 5.3% | 93.1% |
| 16 | 6.0% | 93.3% |
| 17 | 5.5% | 95.0% |
| 18 | 4.3% | 94.8% |

### Example 3—Chemical Compositional Analysis of Other Biomass Species

The components of various substrates before and after pretreatment were determined based on the procedures established by the NERL according the method of Example 2. The tested substrates were pretreated with either (i) 85% ethanol or (ii) 85% 1,4-butanediol, both with the presence of 25 mM H₂SO₄ at a temperature of 170° C. for 60 minutes. The results in Table 3 using 1,4-BDO shows that, with the presently disclosed diol/acid pretreatment step, it is possible to achieve >85% removal of lignin in all of the tested substrates, including pine wood, rice husk and mixed wood, which is a combination of pine wood (softwood) and Eucalyptus (hardwood) in one hour.

Despite using a lower temperature of 170° C., the percentage of lignin removal (yield of lignin) achieved by the present method was higher than that as obtained with the other prior art ethanol pretreatment method. By comparing ethanol and 1,4-BDO pretreatment from Table 2 and Table 3, it is observed that 1,4-BDO is able to achieve better lignin removal percentage and 1,4-BDO is less sensitive to pretreatment severities and substrates as compared to ethanol (1,4-BDO: 87-94%, Ethanol: 70-84%). These results illustrate that optimization and substrate component will not significantly impact the performance of the presently disclosed diol pretreatment step. It is also shown that the presently disclosed diol pretreatment does not require tedious optimization process or selection/purification of substrate, and the method is non-biomass specific.

TABLE 3
Lignin content of various substrates before
and after different pretreatment processes

| | | | | Lignin removal (%) | |
|---|---|---|---|---|---|
| | Lignin content (%) | | | 1,4-BDO | EtOH |
| | Raw | 1,4-BDO | EtOH | pre- | pre- |
| Substrate | substrate | pretreated | pretreated | treatment | treatment |
| Pine wood | 36.1 | 7.3 | 16.4 | 89.9 | 80.8 |
| Rice husk | 27.7 | 7.5 | 13.9 | 87.0 | 75.6 |
| Mixed wood | 30.5 | 5.4 | 13.5 | 92.3 | 84.3 |
| (pine + | | | | | |
| eucalyptus) | | | | | |

In addition, Table 4 shows the lignin content of pine wood and Eucalyptus before and after pretreatment with (i) 85% ethanol or (ii) 85% 1,4-butanediol, both with the presence of 150 mM H₂SO₄, at a temperature 121° C. for 240 minutes. As shown, by subjecting the substrate to the pretreatment step as disclosed, it is possible to achieve >70% removal of lignin from softwood and >80% removal of lignin from hardwood at temperature as low as 121° C. in 4 hours.

The possibly of this relatively low temperature pretreatment step allows the production of energy and chemicals in a cost effective manner, thereby overcoming the major challenges faced by biorefinery concepts known in the art.

TABLE 4
Lignin content of various substrates before
and after different pretreatment processes

| | | | | Lignin removal (%) | |
|---|---|---|---|---|---|
| | Lignin content (%) | | | 1,4-BDO | EtOH |
| | Raw | 1,4-BDO | EtOH | pre- | pre- |
| Substrate | substrate | pretreated | pretreated | treatment | treatment |
| Softwood | 36.1 | 16.9 | 47.5 | 70.4 | 7.8 |
| (Pine) | | | | | |
| Hardwood | 26.1 | 7.4 | 22.9 | 85.7 | 52.2 |
| (eucalyptus) | | | | | |

### Example 4—Physicochemical Properties of Organosolv Lignins

Two-dimensional heteronuclear single quantum coherence (2D HSQC) nuclear magnetic resonance (NMR) analyses were conducted to determine the molecular-scale structure of EtOH and 1,4-BDO pretreated lignin samples.

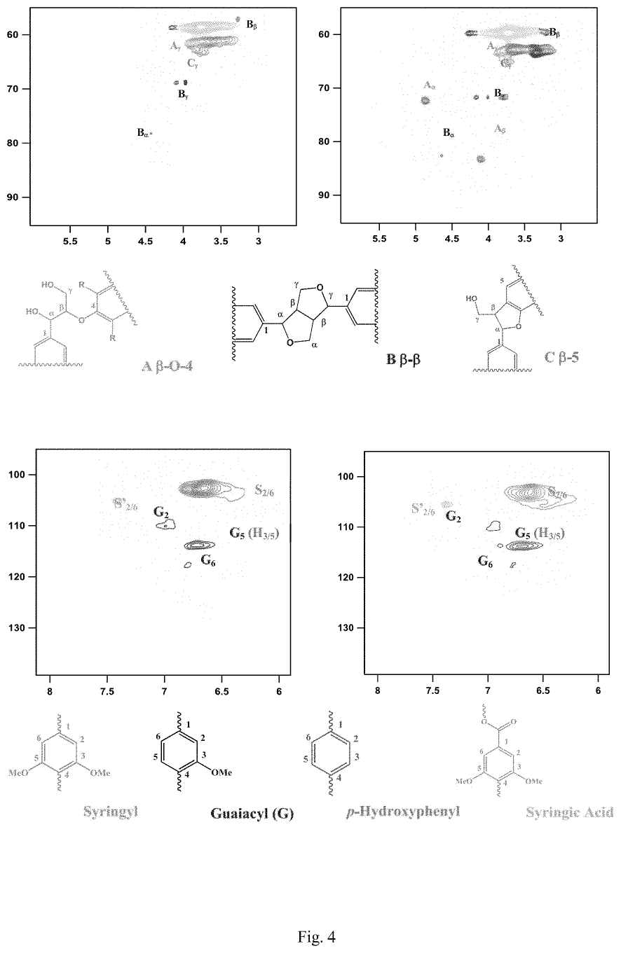

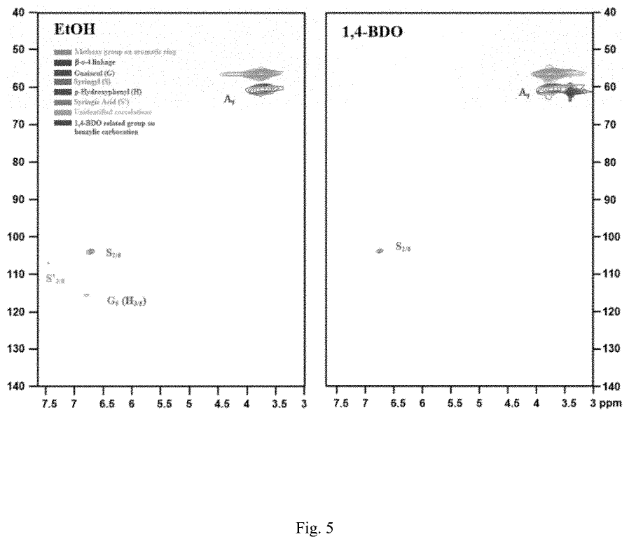

Residual lignins (EtOH-S and 1,4-BDO-S) were prepared and purified according to Meng et al. (Meng, X., et al., Effect of in Vivo Deuteration on Structure of Switchgrass Lignin. ACS Sustainable Chemistry & Engineering, 2017. 5(9): p. 8004-8010 and by using a JEOL ECZR 500 MHz spectrometer equipped with a 5 mm ROYAL probe. The representative 2D HSQC NMR spectra of EtOH and 1,4-BDO pretreated lignin are summarized in FIGS. 4 and 5. In the aliphatic regions of the HSQC NMR spectra, β-O-4, β-5, and β-β were the dominant interunit linkages in lignin samples (FIG. 4 upper row and FIG. 5). A unique signal was discovered in the HSQC spectra of 1,4-BDO pretreated lignin residues and soluble lignin, (Marked in red) which indicated the 1,4-BDO related functional group on the α position of β-O-4 linkage.

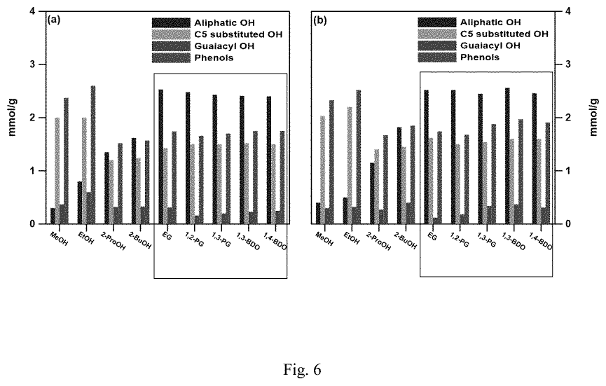

For the quantification of hydroxyl groups in the lignin samples, phosphorylation of each sample was performed with 2-chloro-4,4,5,5-tetramethyl-1,3,2-dioxaphospholane (TMDP) in a solvent of pyridine/CDCl₃ (1.6/1.0, v/v) as described in the previous study of Meng et al. (2017). The quantitative ³¹P NMR spectra were acquired on the JEOL ECZR 500 MHz spectrometer using an inverse-gated decoupling pulse sequence, 900 pulse, 25 s pulse delay with 32 scans. All chemical shifts reported are relative to internal standard at 145 ppm. The quantitative calculation of the hydroxyl groups was based on the amount of the internal standard. The results of ³¹P NMR analyses were demonstrated in FIG. 6.

The amount of phenolic OH groups of lignin from diols pretreated Eucalyptus was approximately 20% higher than MeOH and EtOH lignins (index of preferable structural integrity). In addition, the relatively higher amount of aliphatic OH groups from diols pretreatment further supported the reaction pathway that α position of β-O-4 had been transformed into ether linkages (i.e. diols related functional group).

The weight-averaged molecular weight (M_{w}) and number-averaged molecular weight (Mₙ) of the lignin samples were measured by Gel permeation chromatography (GPC) as described previously (O. Ringena, et a., Size-exclusion chromatography of technical lignins in dimethyl sulfoxide/water and dimethylacetamide, 2006, 1102, 154-163. by size-exclusion separation performed on the HPLC equipped with Agilent PLgel MIXED-B column. Table 4 showed the molecular weight of the seven lignin samples measured by GPC. The trends of molecular weights of dissolved lignin for different solvents consisted with functional group measurements (from PNMR analysis). Both M_{w} and Mₙ of lignin from diols pretreated Eucalyptus were larger than the ones from monohydric alcohols. This means that the quality of the extracted lignin is higher compared with monohydric alcohols.

TABLE 4
Molecular weights and polydispersity
index of organosolv soluble lignin

| Experiment | | | |
|---|---|---|---|
| Condition | M_{w} | Mₙ | PDI |
| 1 | 4391 | 7021 | 1.60 |
| 2 | 4034 | 6510 | 1.61 |
| 3 | 5317 | 7972 | 1.50 |
| 4 | 5179 | 7888 | 1.52 |
| 5 | 5636 | 8461 | 1.50 |
| 6 | 5685 | 8700 | 1.53 |
| 7 | 5161 | 8258 | 1.60 |
| 8 | 5072 | 8391 | 1.65 |
| 9 | 5627 | 8785 | 1.56 |
| 10 | 4722 | 6688 | 1.42 |
| 11 | 4610 | 6641 | 1.44 |
| 12 | 4868 | 6997 | 1.44 |
| 13 | 5161 | 7240 | 1.40 |
| 14 | 5299 | 8171 | 1.54 |
| 15 | 5036 | 7412 | 1.53 |
| 16 | 5153 | 7753 | 1.47 |
| 17 | 5150 | 7798 | 1.51 |
| 18 | 5045 | 7820 | 1.55 |

### Example 5—Enzymatic Hydrolysis

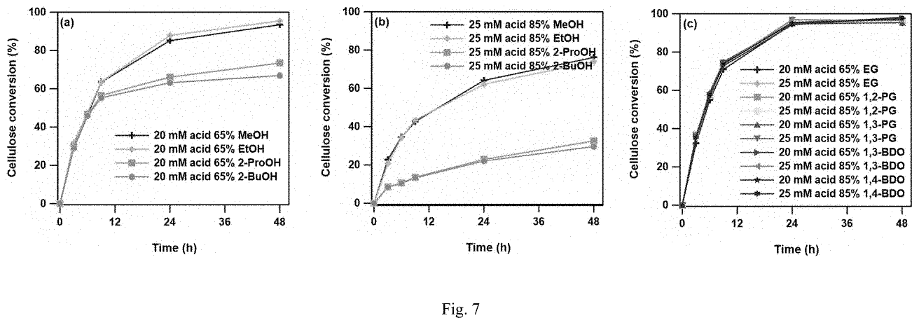

Enzymatic hydrolysis was conducted at 2% (w/v) solid loading in acetate buffer (50 mM, pH 4.8) with an enzyme loading of 7.5 FPU/g glucan in a 500 mL Erlenmeyer flask. The mixture was incubated in a rotary shaker at 50° C. and 150 rpm for 72 h. A 1.00 ml sampling of supernatant was collected at 3, 6, 9, 24, 48, 72 h for sugar analysis. The digestibility of cellulose in the pretreated substrates from Example 1 were demonstrated in FIG. 7.

At a pretreatment severity of 20 mM acid, 65% organosolv, cellulose contents of MeOH, and EtOH pretreated Eucalyptus were enriched to more than 90% (due to lignin and hemicelluloses removal), while cellulose contents of MeOH, and EtOH pretreated Eucalyptus reduced to below 80% when pretreatment severity increased to 25 mM, 85% organosolv. For 2-ProOH and 2-Bu pretreated substrates, cellulose contents dropped even more significantly to under 40%, when pretreatment condition was changed to 25 mM, 85% organosolv. However, this phenomenon was not observed in organosolv pretreatment with diols. Cellulose conversions were found to be over 90% at both pretreatment severities. These results show that, as compared to extracts pretreated with other known organosolv pretreatment methods, the cellulose-enriched extract pretreated with diols consistently displays high enzymatic digestibilities, regardless of the pretreatment conditions. The results also demonstrate that the pretreatment method is highly adaptable and can be used with minimal optimization.

As demonstrated by the examples, the presently disclosed new pretreatment method makes use of non-toxic, high boiling point solvent and low pretreatment temperature, whilst producing significantly high yield of lignin from the liquor and glucose from the cellulose-enriched product. It is therefore expected that the process decreases the overall cost and the environmental impact of biorefinery systems, making it a feasible option for commercialization.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. Rather, the described features and steps are disclosed as examples of components of systems and methods within the scope of the appended claims.

1. A method of treating lignocellulose biomass for extraction of target chemical products therefrom comprising:
a. pretreating the lignocellulose biomass with at least one diol in the presence of at least one acid, and optionally, water; and
b. separating the pretreated mixture into solid and liquid products.

2. The method according to claim 1, wherein the pretreatment step is performed at or below about 170° C.

3. The method according to claim 1, wherein the pretreatment step is performed at a temperature range of between about 100° C. to about 170° C., preferably under atmospheric pressure to approximate 200 kPa.

4. The method according to claim 1, wherein the lignocellulosic biomass is selected from the group consisting of grasses, straws, husk, bagasse, hardwood, softwood, and combinations thereof.

5. The method according to claim 1, wherein the lignocellulosic biomass is comprised of a plurality of plant species.

6. The method according to claim 1, wherein the at least one diol is selected from the group consisting of ethylene glycol, 1,2-propanediol, 1,3-propanediol, 1,3-butanediol, and 1,4-butanediol.

7. The method according to claim 4, wherein the at least one diol is 1,4-butanediol.

8. The method according to claim 1, wherein the at least one acid is selected from the group consisting hydrochloric acid, formic acid, acetic acid and phosphoric acid and sulphuric acid.

9. The method according to claim 6, wherein the at least one acid is sulfuric acid.

10. The method according to claim 1, wherein the concentration of acid is 1 mM-200 mM.

11. The method according to claim 1, wherein the ratio of diol to biomass is 1:7 or lower.

12. The method according to claim 1, wherein the ratio of water to diol is 1:100 or lower.

13. The method according to claim 1 further comprising extracting lignin from the liquid product.

14. The method according to claim 1, further comprising enzymatic hydrolysis of the solid product, and optionally fermentation of the sugars obtained from the hydrolysis.

15. The method according to claim 14, wherein the enzymatic hydrolysis is performed with one or more enzymes selected from the group consisting of cellulose, endo-cellulase, exo-cellulase, beta-glucosidase, cellobiase, oxidative cellulase, cellulose phosphorylases, hemicellulases, xylanases, arabinases, mannanases.

16. The method according to claim 14, further comprising fractional distillation of the fermentation product to recover target products.

17. The method according to claim 14, wherein the pretreatment step, the enzymatic hydrolysis step and the fermentation steps take place in the same reactor.

18. A system for treating one or more lignocellulose biomass according to the method of claim 1, wherein the system comprises a reactor for carrying out the pretreatment step wherein said reactor is made of a material selected from plastic, glass and metal.

19. The system according to claim 18, wherein the system further comprises:
a. Means for separating the pretreated mixture to produce a cellulose-enriched product stream and lignin-enriched product stream;
b. Optionally, means for size reduction;
c. Optionally, means for separating purified lignin,
d. Optionally, a saccharification and fermentation unit; and
e. Optionally, a fractional distillation unit.

20. The system according to claim 18, wherein the system comprises a single unit for pretreatment, enzymatic hydrolysis and fermentation.
